(19) United States
(12) Patent Application Publication
(10) Pub. No.: US 2016/0008090 A1
(43) Pub. Date: Jan. 14, 2016

(54) PUNCTURE NEEDLE GUIDING DEVICE AND IMAGE DATA FORMING APPARATUS

(71) Applicant: TERUMO KABUSHIKI KAISHA, Tokyo (JP)

(72) Inventors: Nao YOKOI, Sunnyvale, CA (US); Masakatsu KAWAURA, Sunnyvale, CA (US); Shigeki ARIURA, Ebina-shi (JP)

(73) Assignee: TERUMO KABUSHIKI KAISHA, Tokyo (JP)

(21) Appl. No.: 14/865,426

(22) Filed: Sep. 25, 2015

Related U.S. Application Data
(63) Continuation of application No. PCT/JP2013/059891, filed on Apr. 1, 2013.

(51) Int. Cl. A61B 19/00; A61F 2/00

(52) U.S. Cl. CPC A61B 19/5244; A61F 2/0045; A61B 2019/5263

(57) ABSTRACT

A puncture needle guiding device is disclosed, which can include a casing; an ultrasound transmission-reception unit which is disposed inside the casing and which transmits and receives ultrasound; and a guide groove which is provided in the casing and which guides a curved puncture needle adapted to puncture a subject body.

### CROSS-REFERENCES TO RELATED APPLICATIONS

This application is a continuation of International Application No. PCT/JP2013/059891 filed on Apr. 1, 2013, the entire content of which is incorporated herein by reference.

### TECHNICAL FIELD

The present disclosure generally relates to a puncture needle guiding device and an image data forming apparatus.

### BACKGROUND DISCUSSION

If a person suffers from a urinary incontinence, for example, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth or the like.

For the treatment of urinary incontinence, a surgical treatment is effective, in which there is used, for example, a belt-shaped biological tissue-supporting indwelling article called “sling.” The sling is indwelled inside the body and the urethra is supported by the sling (see, for example, Japanese Patent Laid-open No. 2010-99499). In order to indwell the sling inside the body, an operator would incise the vagina with a surgical knife, dissect the biological tissue between the urethra and vagina, and make the dissected region and the outside communicate with each other through obturator foramens by use of a puncture needle. Then, in this state, the sling is indwelled into the body.

If the vagina is incised once, however, a situation may occur that the sling is exposed to the inside of the vagina from a wound caused by the incision of the vagina, and complications may be caused by an infection from the wound or the like. Further, since the vagina is incised, the invasiveness of the incision can be relatively great and the burden relatively high on the patient. Further, the urethra may be damaged in the course of the procedure by the operator. In addition, the fingertip of the operator may be damaged or injured.

### SUMMARY

A puncture needle guiding device and an image data forming apparatus are disclosed by which puncturing by a puncture needle could be performed accurately.

A puncture needle guiding device is disclosed, which can include a casing; an ultrasound transmission-reception unit which is disposed inside the casing and which transmits and receives ultrasound; and a guide section which is provided in the casing and which guides a curved puncture needle adapted to puncture a subject body.

In the puncture needle guiding device, preferably, the puncture needle is rotationally movable.

In the puncture needle guiding device, the puncture needle may be rotationally movable so as to describe an arc.

In the puncture needle guiding device, preferably, the puncture needle is arc-shaped.

An image data forming apparatus is disclosed, which can include a puncture needle guiding device including a casing, an ultrasound transmission-reception unit which is disposed inside the casing and which transmits and receives ultrasound, and a guide section which is provided in the casing and which guides a curved puncture needle adapted to puncture a subject body; and a control unit which forms image data for displaying on a display unit an image based on information obtained by the ultrasound transmission-reception unit.

In the image data forming apparatus, preferably, the control unit forms image data for displaying on the display unit both the image based on the information obtained by the ultrasound transmission-reception unit and an image including a puncture path for a needle tip of the puncture needle.

In the image data forming apparatus, preferably, the image data further includes a positioning marker for positioning the puncture needle guiding device such that when a body lumen or cavity and the positioning marker are matched to each other on the display unit, the puncture needle is guided by the guide section so as to avoid the body lumen or cavity.

In the image data forming apparatus, the casing may be used in cooperation with an insertion section, which is inserted into a living body or into a body lumen or cavity.

In the image data forming apparatus, preferably, the ultrasound transmission-reception unit is used for treatment of a disorder in a pelvic organ and is so disposed as to make contact with a body surface.

In the image data forming apparatus, the guide section may include a groove.

In the image data forming apparatus, preferably, the guide section can include a rotating shaft of the puncture needle, which is rotationally movable.

In the image data forming apparatus, the guide section may be freely detachable in relation to the casing.

In the image data forming apparatus, preferably, an operation unit operated to rotate the puncture needle is connected to the puncture needle.

In the image data forming apparatus, preferably, the body lumen or cavity is a urethra, and the puncture needle guiding device is so configured that the insertion section and the casing can be interlocked to each other.

A method is disclosed of guiding a puncture needle during formation of a path in a living body, the method comprising: transmitting and receiving ultrasound from an ultrasound transmission-reception unit which is disposed inside a casing; guiding a curved puncture needle with a guide section in the casing; and puncturing the living body with the puncture needle.

According to the described need and the aforementioned configurations, the puncture needle can be guided by the guide section and, therefore, the puncture by the puncture needle can be performed relatively smoothly. In addition, since the puncture by the puncture needle can be conducted relatively smoothly, the puncture by the puncture needle can be carried out with a reduced force, so that the safety of the surgical treatment can be enhanced. In addition, where an image based on the information obtained by the ultrasound transmission-reception unit is displayed on the display unit such as a monitor, the puncture by the puncture needle can be performed on a visible basis. Therefore, the puncture by the puncture needle can be carried out relatively accurately and safely. For example, where a puncture path for a needle tip of the puncture needle is displayed in the state of being superimposed on the ultrasonic image displayed on the monitor, the puncture needle guiding device can be easily disposed in a suitable position and, accordingly, the puncture by the puncture needle can be performed relatively accurately and safely.

### BRIEF DESCRIPTION OF THE DRAWINGS

FIG. 1A is a perspective view of a puncture member in accordance with an exemplary embodiment;

FIG. 1B is a plan view of the puncture member in accordance with an exemplary embodiment;

FIG. 2 is a perspective view of an implant in accordance with an exemplary embodiment;

FIG. 3 is a block diagram showing an image data forming apparatus according to a first exemplary embodiment of the present disclosure;

FIG. 4 is a partially sectional plan view of a puncture needle guiding device possessed by the image data forming apparatus shown in FIG. 3;

FIG. 5 is a plan view of the puncture needle guiding device shown in FIG. 4;

FIG. 6A is a bottom plan view of the puncture needle guiding device shown in FIG. 5;

FIG. 6B is a side view of the puncture needle guiding device shown in FIG. 5;

FIG. 7 depicts an image displayed on a display unit possessed by the image data forming apparatus shown in FIG. 3;

FIG. 8 is a drawing for explaining an operating method of the puncture needle guiding device shown in FIG. 4;

FIG. 9 is a drawing for explaining the operating method of the puncture needle guiding device shown in FIG. 4;

FIG. 10 is a drawing for explaining the operating method of the puncture needle guiding device shown in FIG. 4;

FIG. 11 is a perspective view of a puncture needle guiding device possessed by an image data forming apparatus according to a second exemplary embodiment of the present disclosure;

FIG. 12 is a plan view showing a modification of the puncture needle guiding device shown in FIG. 11;

FIG. 13 is a sectional view showing a modification of the puncture needle guiding device shown in FIG. 11;

FIG. 14 is a side view of a puncture apparatus;

FIG. 15 is a sectional view taken along line XV-XV of FIG. 14; and

FIG. 16 is a drawing showing a state in which the puncture apparatus shown in FIG. 14 and a puncture needle guiding device are interlocked to each other.

### DETAILED DESCRIPTION

A puncture needle guiding device and an image data forming apparatus according to one mode of the present disclosure will be described in detail below, referring to some preferred embodiments illustrated by way of example in the accompanying drawings.

In accordance with an exemplary embodiment, a first exemplary embodiment of the puncture needle guiding device and the image data forming apparatus of the present disclosure will be described.

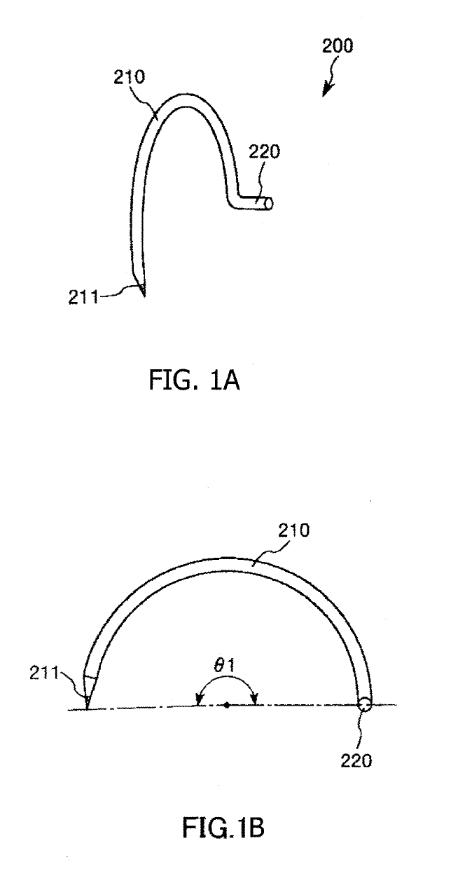

In the following, for convenience of explanation, the upper side in FIGS. 1A to 12 will be referred to as “proximal” side, and the lower side as “distal” side. In addition, FIGS. 1A to 4 show the puncture needle guiding device or the image data forming apparatus in its state of being not yet put to use, which state will be hereinafter referred to also as an “initial state” for convenience of explanation.

First, a medical device to be used with an image data forming apparatus 1 in a surgical treatment will be described.

A puncture member 200 shown in FIGS. 1A and 1B can include a curved puncture needle 210 adapted to puncture biological tissue (subject body), and an operation unit (grasping unit) 220 which is connected to the puncture needle 210 and rotates the puncture needle 210.

In accordance with an exemplary embodiment, the puncture needle 210 is curved in an arc shape, and has a sharp needle tip at the distal end of the puncture needle 210. The puncture needle 210 can be formed in an arc shape with a constant curvature, which helps permit the puncture needle 210 to be smoothly advanced within a guide groove 361 (described later) and to be smoothly guided by the guide groove 361. In addition, the provision of the operation unit 220 can help ensure that an operation of rotating the puncture needle 210 can be performed relatively easily and that a procedure, which will be described later, can be carried out suitably. The puncture member 200 as above is so configured as to puncture biological tissue in the vicinity of a urethra and a vagina, by way of a part different from the urethra and the vagina.

The center angle θ1 of the arc of the puncture needle 210 is not particularly limited, and is appropriately set according to various conditions. The center angle θ1 can be set so that at the time of puncturing biological tissue of a patient by the puncture needle 210, the puncture needle 210 can enter the living body via an inguinal region on one side of the patient, pass a region on the lower side of the urethra and exit the living body via an inguinal region on the other side. For example, the center angle θ1 can be, preferably 150 to 270°, more preferably 170 to 250°, and further preferably 190 to 230°. With the center angle θ1 set within such a range, the aforementioned puncture can be carried out reliably.

The puncture needle 210 is formed with a through-hole 211 in a distal portion of the puncture needle 210. The through-hole 211 is for inserting one of strings 310 and 320 possessed by an implant 300 described later therein and for retaining the string in a detachable state.

The material constituting the puncture member 200 is not specifically restricted. For example, various metallic materials such as stainless steel, aluminum or aluminum alloys, titanium or titanium alloys can be used.

The implant is an embeddable instrument for treatment of female urinary incontinence, for example, for supporting the urethra. For example, the implant can be an instrument, which when the urethra is going to move toward the vaginal wall side, supports the urethra so as to restrict its movement in the direction of coming away from the vaginal wall. As the implant, there can be used a flexible elongated body, for example.

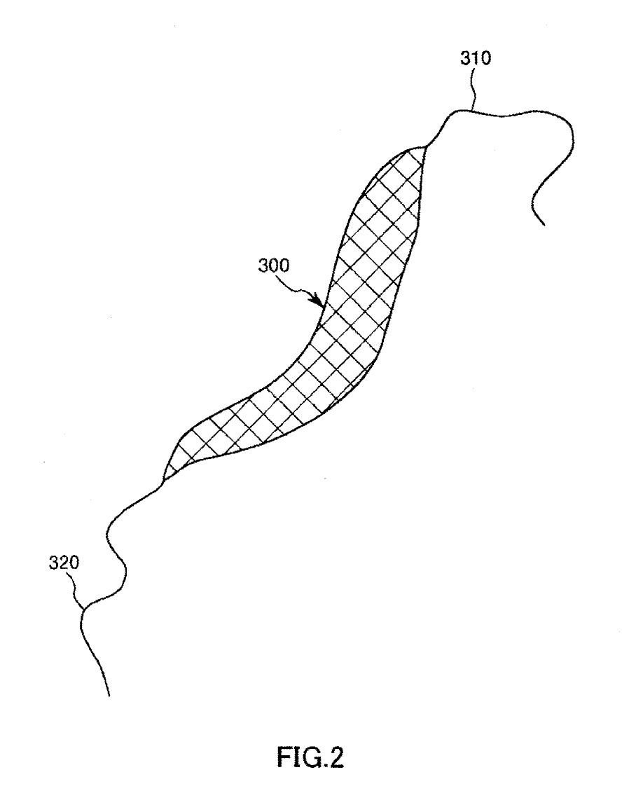

The implant 300 shown in FIG. 2 is net-like in form, and has a belt-like overall shape. The implant 300 is called “sling.” Note that the implant 300 may be composed of a network (lattice)-formed knitted body knitted by causing linear (filamentous) elements to intersect, for example, a network-formed braiding. Examples of the linear element include those, which can be circular in cross section, and those, which can be flat shaped in cross section, for example, belt-shaped (ribbon-shaped) ones. An end portion on one side of the string 310 can be fixed to one end of the implant 300, and an end portion on one side of the string 320 can be fixed to the other end of the implant 300.

The material constituting the implant 300 is not particularly limited. Examples of the material usable for the implant 300 include various biocompatible resin materials, fibers and the like, such as polypropylene, polyester and nylon. In addition, the materials constituting the strings 310 and 320 are not specifically restricted. Examples of the materials usable for the strings 310 and 320 can include various biocompatible resin materials, fibers and the like, such as polypropylene.

Note that the implant 300 is naturally not limited to the network-formed one, so long as it can produce the same or equivalent effect.

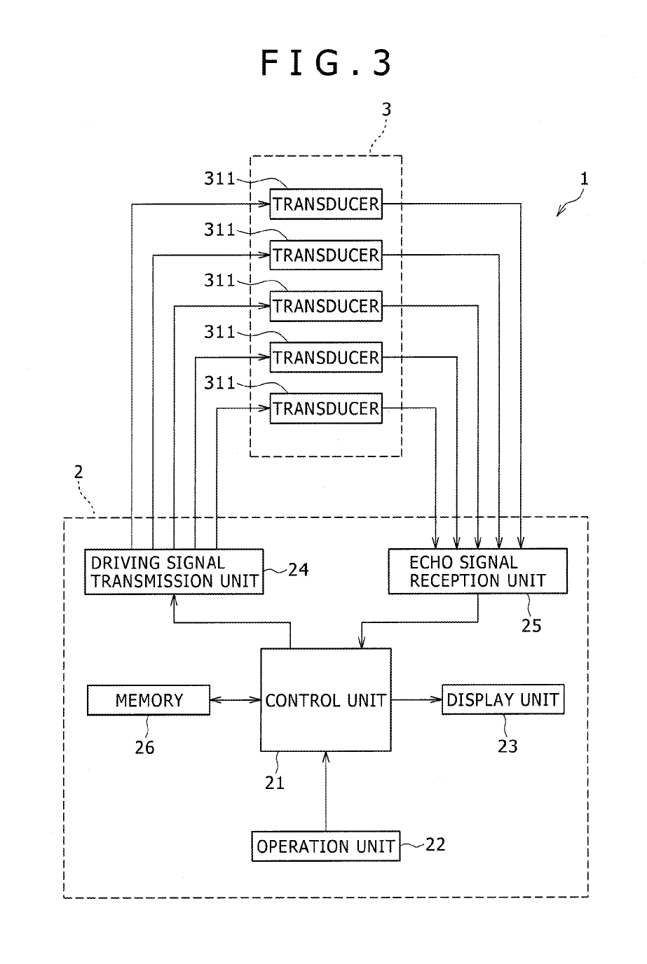

The image data forming apparatus will be described below. The image data forming apparatus 1 shown in FIG. 3 can include an apparatus main body 2 and a puncture needle guiding device 3.

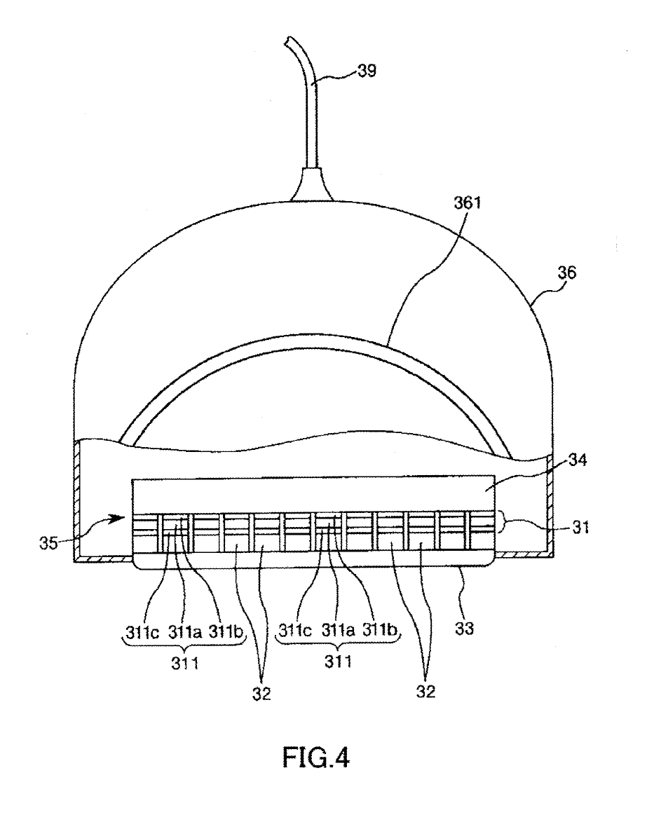

The puncture needle guiding device 3 is a transabdominal ultrasonic probe. As shown in FIG. 4, the puncture needle guiding device 3 can include an ultrasound transmission-reception unit 35 including a transducer array 31, acoustic matching layers 32, an acoustic lens 33, and a backing material 34; a casing 36 accommodating the ultrasound transmission-reception unit 35; and a cable 39 extending from the casing 36.

The transducer array 31 transmits and receives ultrasounds. The transducer array 31 can include a plurality of transducers 311 arrayed in a straight row. Each of the plurality of transducers 311 has a configuration in which a piezoelectric body 311a formed of lead zirconate titanate (PZT), polyvinylidene fluoride (PVDF), or quartz is sandwiched between a pair of electrodes 311b and 311c. When an alternating current (AC) voltage having a specified frequency is impressed between the pair of electrodes 311b and 311c, the piezoelectric body 311a is vibrated to generate ultrasound. On the contrary, when the piezoelectric body 311a is vibrated by an external force (ultrasound), a voltage according to the vibration is generated between the pair of electrodes 311b and 311c.

The acoustic lens 33 is located on the front side (the lower side in FIG. 4) of the transducer array 31. The acoustic lens 33 converges the ultrasound generated from the transducer array by utilizing refraction, which can help enhance the resolving power of the puncture needle guiding device 3. In addition, the acoustic lens 33 is also a part which is exposed to the outside from the distal end (lower end) of the casing 36 and which comes into contact with the body surface of the subject body at the time of examination. Therefore, the acoustic lens 33 also has a function to reduce friction with the living body. The acoustic lens 33 is convexed in shape, and is formed from a material (e.g., silicone rubber) in which ultrasounds are propagated slower than in the living body. With the acoustic lens 33 (ultrasound transmission-reception unit 35) thus put in contact with the body surface of the subject body, the transmission of ultrasound to the inside of the living body can be performed efficiently.

The acoustic matching layers 32 can be provided between the transducer array 31 and the acoustic lens 33. The acoustic matching layer 32 has a function to reduce the difference in impedance between each transducer 311 and the living body. By the acoustic matching layer 32, ultrasound propagation efficiency can be enhanced and the ultrasound can be transmitted and received efficiently. The acoustic matching layer 32 may be composed, for example, of a laminate of a plurality of layers each formed from a resin material.

The backing material 34 is disposed on the back side of the transducer array 31. The backing material 34 restrains transmission of ultrasound to the back side, and shortens the pulse width of the ultrasound. By this, the distance resolving power of the puncture needle guiding device 3 can be enhanced. The backing material 34 can be generally formed from one of various resin materials such as rubber materials.

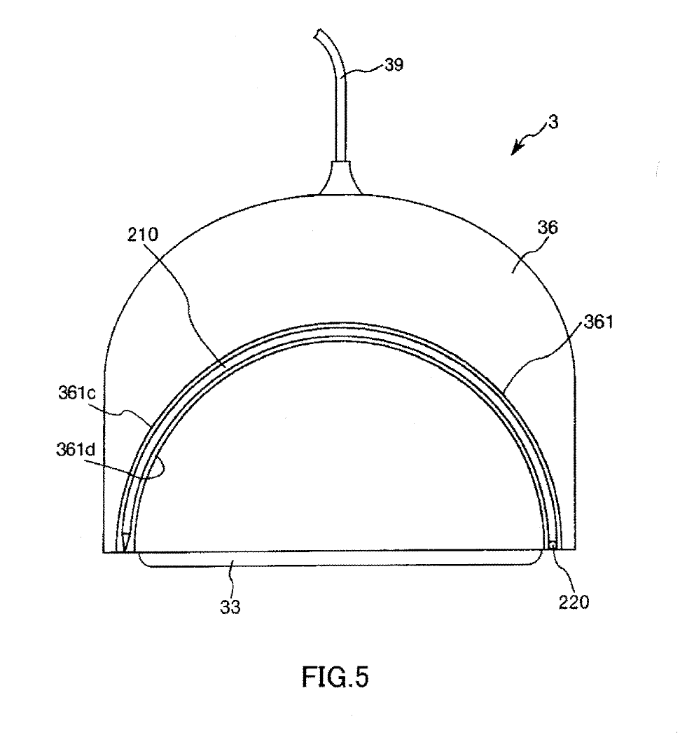

As shown in FIG. 5, the casing 36 is formed with the guide groove 361 (guide section) which guides the puncture needle 210. The guide groove 361 can have restriction units 361c and 361d, which are located on both sides in the width direction of the puncture needle 210 and which restricts movement of the puncture needle 210 in other directions than the extending direction thereof (for example, movement in the width direction). With the guide section composed of the guide groove 361, the configuration of the guide section can be simplified, and the puncture needle 210 can be guided relatively smoothly.

The guide groove 361 is formed in an arc shape, which corresponds to the puncture needle 210. Therefore, the guide groove 361 can guide the puncture needle 210 in the manner of rotationally moving the puncture needle 210 along the arc in the extending direction thereof. Consequently, the guiding of the puncture needle 210 can be conducted smoothly. Note that the shape of the guide groove 361 is not particularly limited, so long as the puncture needle 210 can be guided thereby. For example, a configuration may be adopted in which pairs of pins (restriction units) disposed so as to clamp (or positionally restrict) the puncture needle 210 in the width direction is disposed at a plurality of positions in the extending direction of the puncture needle 210.

An opening of the guide groove 361 is designed, for example, to have a width equal to or slightly greater than the diameter of the puncture needle 210 so that the puncture needle 210 can be disposed into the guide groove 361 by way of the opening, which helps enable the puncture needle 210 to be mounted into the guide groove 361 relatively easily. Note that the width of the opening may be designed to be slightly smaller than the diameter of the puncture needle 210, contrary to the above-mentioned. In the latter case, also, the puncture needle 210 can be disposed into the guide groove 361, by pushing the puncture needle 210 into the guide groove 361. In addition, in the state where the puncture needle 210 is disposed in the guide groove 361, unintentional dislodgement of the puncture needle 210 via the opening can be prevented. Accordingly, the guide groove 361 helps enable smoother guiding of the puncture needle 210.

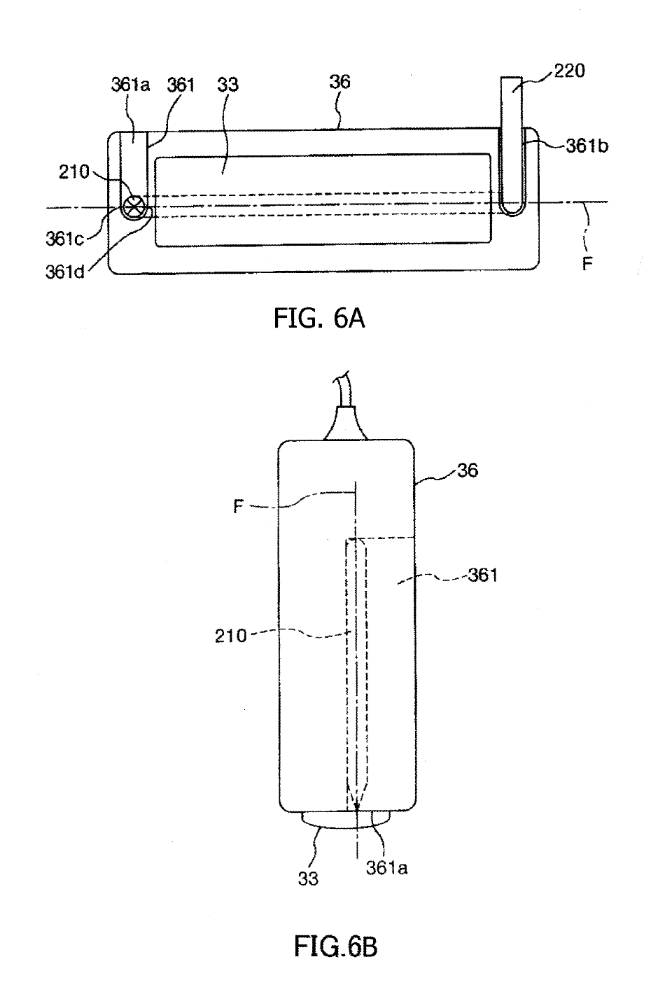

In addition, as shown in FIGS. 6A and 6B, the guide groove 361 can be formed so as to overlap with the acoustic lens 33 (the transducer array 31). In addition, the guide groove 361 can be configured that the puncture needle 210 can be rotationally moved in the manner of surrounding the ultrasound transmission direction. In accordance with an exemplary embodiment, for example, the guide groove 361 can be said to be configured to guide the needle tip of the puncture needle 210 so as to cross the region where the ultrasound is transmitted from the ultrasound transmission-reception unit 35; for example, the guide groove 361 can be configured to guide the puncture needle 210 so as to stride across at least a part of the ultrasound transmission-reception unit 35 in a planar view as viewed from the direction orthogonal to a plane F containing the puncture path of the needle tip of the puncture needle 210. Furthermore, the guide groove 361 can be configured such that the puncture path (plane F) of the needle tip of the puncture needle 210 guided by the guide groove 361 intersects the acoustic lens 33 and is contained in the ultrasound transmission region, which can help ensure that the puncture path of the puncture needle 210 can be expressed in the form of an image, which can be reliably visualized.

In accordance with an exemplary embodiment, both ends 361a and 361b of the guide groove 361 are opening in the distal surface of the casing 36, on both sides of the acoustic lens 33. With the guide groove 361 disposed in this way, the puncture needle 210 can be made to puncture a part expressed in the form of an image by the image data forming apparatus 1 (a part displayed on the display unit 23). In accordance with an exemplary embodiment, for example, the puncture path of the puncture needle 210 can be expressed in the form of an image. Accordingly, the puncture by the puncture needle 210 can be performed on a visible basis and in a relatively accurate and safe manner.

As shown in FIG. 3, the apparatus main body 2 can include a control unit 21, an operation unit 22 for performing various operations, the display unit 23 for displaying each information, a driving signal transmission unit 24 for transmitting driving signals to the transducers 311, an echo signal reception unit 25 for receiving voltages (echo signals) generated from the transducers 311, and a memory 26. The puncture needle guiding device 3 is electrically connected to the apparatus main body 2 through the cable 39. Note that at least one of the units included in the apparatus main body 2 (for example, the driving signal transmission unit 24 or the echo signal reception unit 25) may be provided in the puncture needle guiding device 3.

The control unit 21 is composed, for example, of a personal computer. In accordance with an exemplary embodiment, the control unit 21 receives input signals from the operation unit 22 and controls the whole part of the apparatus, such as the display unit 23, the driving signal transmission unit 24, and the echo signal reception unit 25. The display unit 23 is not particularly limited, so long as it can display images; for example, a liquid crystal display, a cathode-ray tube (CRT) or the like may be used.

The driving signal transmission unit 24 transmits to the transducers 311 electrical signals for transmitting ultrasound from the transducers 311. The driving signal transmission unit 24 can control the driving of the transducers 311 independently. In accordance with an exemplary embodiment, for example, the echo signal reception unit 25 receives echo signals, which are generated from the transducers 31 through reception of the ultrasounds returning after being reflected inside the living body. The echo signals from the transducers 311 thus received are outputted to the control unit 21.

The control unit 21 controls the driving signal transmission unit 24 and the echo signal reception unit 25, for performing transmission and reception of ultrasounds by the transducers 311, and forms image data for displaying an ultrasonic image on the display unit 23, on the basis of the signals transmitted from the echo signal reception unit 25 (the information obtained from the transducers 311). Then, the control unit 21 displays an ultrasonic image on the display unit 23 on the basis of the image data thus formed.

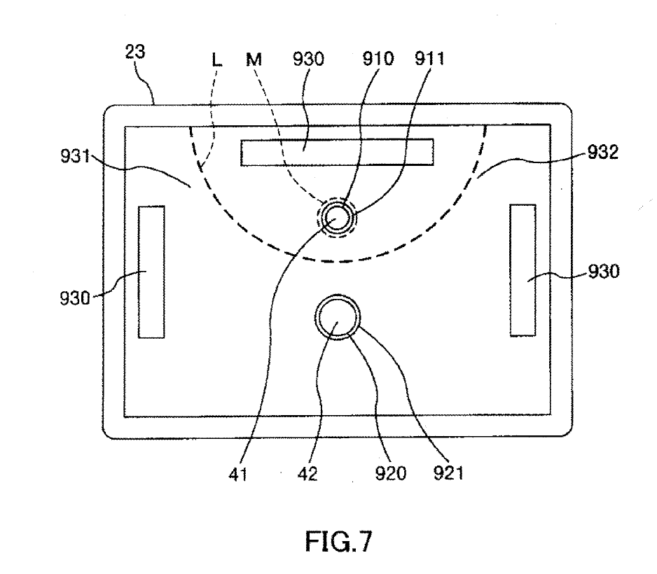

The ultrasonic image is an image obtained by transmitting ultrasounds from the transducers 311, measuring the distance from each transducer 311 to an object on which the ultrasound is reflected, from the time elapsed until the reflected wave returns to the relevant transducer 311, and visualizing the object, while taking into account the intensity of the reflected wave and the like. As shown in FIG. 7, the ultrasonic image can include the patient's urethra 910 (urethral wall 911), vagina 920 (vaginal wall 921), and pelvis 930.

Here, in order to express the urethra 910 and the vagina 920 clearly, a urethral ultrasonic marker (insertion section) 41 may be inserted into the urethra 910, and a vaginal ultrasonic marker (insertion section) 42 may be inserted into the vagina 920. By thus using the puncture needle guiding device 3 in cooperation with the urethral ultrasonic marker 41 and the vaginal ultrasonic marker 42, the puncture by the puncture needle 210 can be performed relatively safely. Note that the term “cooperation” means that the puncture needle guiding device 3 and the markers 41 and 42 cooperate with each other, by exhibiting their functions in combination, in placing the implant 300 indwelling inside a living body.

The urethral ultrasonic marker 41 and the vaginal ultrasonic marker 42 each have a suitable shape (for example, a bar- or rod-like shape with its distal end rounded) for insertion into the urethra 910 and the vagina 920, respectively. The materials constituting the urethral ultrasonic marker 41 and the vaginal ultrasonic marker 42 are not particularly limited, so long as they can be visually recognized in an ultrasonic image. For example, a metallic material such as stainless steel, aluminum or aluminum alloys, titanium or titanium alloys, may be used, with its surface provided with a plurality of minute projections and recesses. For example, when the surface of a part formed of a metallic material is formed with a plurality of minute projections and recesses, the part formed with the plurality of projections and recesses can be detected ultrasonically. Alternatively, a urethral ultrasonic marker may be disposed on a urethral-insertion tool to be inserted into the urethra 910, and a vaginal ultrasonic marker may be disposed on a vaginal-insertion tool to be inserted into the vagina 920.

Note that the urethral ultrasonic marker 41 and the vaginal ultrasonic marker 42 are not limited to the bar- or rod-like ones, and may be composed of ultrasonically visible implants capable of being set indwelling in the urethra and the vagina, respectively, or may be composed of an ultrasonic-basis contrast medium composing of a liquid or a gas.

In addition, the control unit 21 obtains a puncture path (predicted path) L for the needle tip of the puncture needle 210 at the time of puncturing biological tissue by the puncture member 200, and forms image data for displaying an image of the puncture path L on the display unit 23. Further, the control unit 21 forms image data for displaying on the display unit 23 a positioning marker M used for positioning the puncture needle guiding device 3 relative to the living body. Then, the control unit 21 displays in a superimposed state both the image of the puncture path L and the image of the positioning marker M on the display unit 23. The puncture path L and the positioning marker M can be made clear by indicating them in broken lines or by indicating them in different colors from the color of other parts.

The positioning marker M can be, for example, for adjusting the urethra thereto; in this case, when the urethra is adjusted to the positioning marker M on the display unit 23, the puncture path L is so positioned as to pass between the urethra 910 and the vagina 920. In accordance with an exemplary embodiment, for example, the puncture needle 210 is guided so as to avoid the positioning marker M. Therefore, with the positioning marker M adjusted to a part, which should not be punctured, erroneous puncture by the puncture needle 210 can be prevented. As a result, erroneous puncture of the urethra 910 or the vagina 920 by the puncture needle 210 can be effectively prevented from occurring.

In accordance with an exemplary embodiment, for example, by setting the positioning marker M to be a marker for the urethra 910 which should not be punctured, erroneous puncture of the urethra 910 can reliably be prevented from occurring. The positioning marker M may not be limited to the marker for positioning the urethra 910, and may also be a marker for positioning the vagina 920.

The puncture path L can be obtained, for example, on the basis of the radius of the arc of the puncture needle 210 and the positional relation between the transducer array 31 and the puncture needle 210. The information on the radius of the puncture needle 210 and information on the positional relation between the transducer array 31 and the puncture needle 210 are preliminarily stored in the memory 26. On the basis of the information stored in the memory 26, the control unit 21 forms the image data of the puncture path L. In addition, the positioning marker M can be obtained, for example, on the basis of the positional relation between the urethra 910 and the vagina 920 analyzed from the ultrasonic image obtained from the signals from the puncture needle guiding device 3 and the puncture path L obtained in the above-mentioned manner.

A method of using the image data forming apparatus 1 will be described below. Here, description will be made of a procedure until the implant 300 for treatment of female urinary incontinence is embedded in a living body by use of the image data forming apparatus 1 and the puncture member 200.

First, the urethral ultrasonic marker 41 is inserted into the urethra 910 of a patient, and the vaginal ultrasonic marker 42 into the vagina 920, as required. Next, the image data forming apparatus 1 is started, and an ultrasonic image of the patient's lower abdominal region is displayed on the display unit 23 by use of the puncture needle guiding device 3. As shown in FIG. 7, on the display unit 23 are displayed the urethra 910 (the urethral ultrasonic marker 41), the vagina 920 (the vaginal ultrasonic marker 42), the pelvis 930, the puncture path L for the puncture needle 210, and the positioning marker M, etc. Looking at the image displayed on the display unit 23, the operator adjusts the urethra 910 to the positioning marker M. Consequently, as shown in FIG. 7, the puncture path L is put into a state of passing an obturator foramen 931, between the urethra 910 and the vagina 920, and an obturator foramen 932, and being sufficiently spaced from the urethral wall 911 and from the vaginal wall 921.

Note that if the puncture path L is in contact with the pelvis 930 or if the puncture path L is too proximate to or overlapping with the urethral wall 911 or the vaginal wall 921, then the position of the puncture needle guiding device 3 can be finely regulated so that the puncture path L is positioned properly. In this instance, where the urethra 910 and the vagina 920 are too close to each other to obtain a suitable puncture path L, the urethra 910 and the vagina 920 may be spaced apart from each other by an operation such as to move at least one of the urethral ultrasonic marker 41 and the vaginal ultrasonic marker 42 in a direction for spacing away from the other.

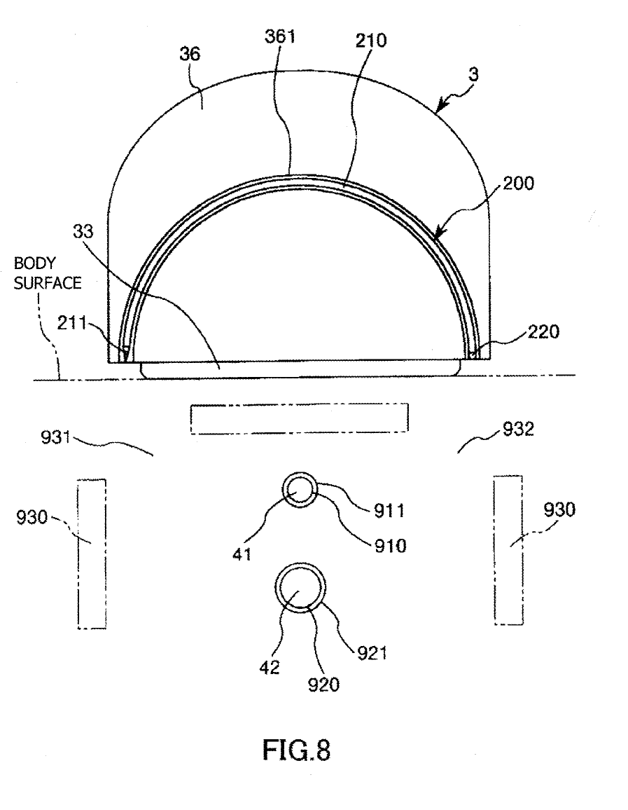

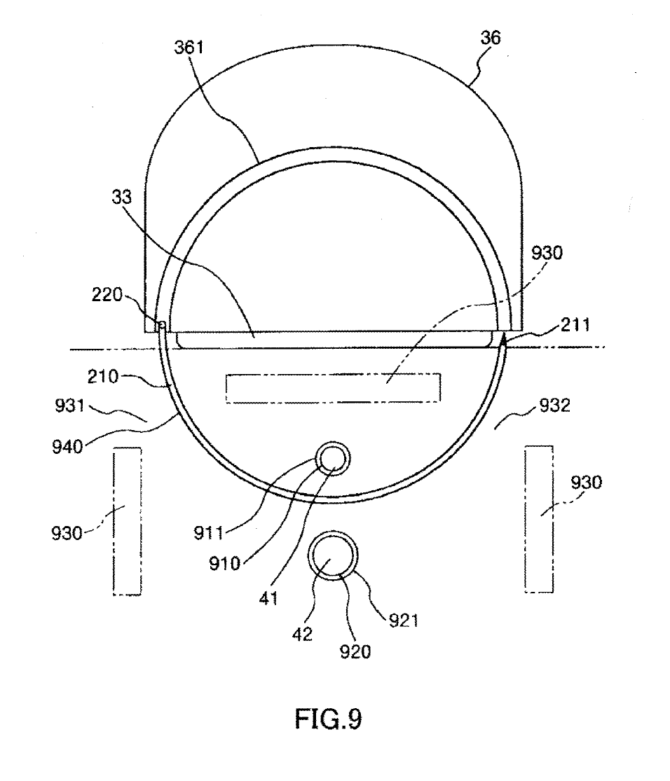

Subsequently, as shown in FIG. 8, while maintaining the proper puncture path L, the puncture member 200 is disposed into the guide groove 361 of the puncture needle guiding device 3. Note that the puncture member 200 may be preliminarily disposed in the guide groove 361. Then, a puncturing operation is conducted by the puncture member 200. For example, with the operation unit 220 grasped, the puncture member 200 is rotated counterclockwise in FIG. 8. By this operation, as shown in FIG. 9, the needle tip of the puncture needle 210 is made to enter the living body by puncturing the body surface in an inguinal region on the left side of the patient or at a part in the vicinity of the inguinal region, to sequentially pass the obturator foramen 931, the lower side of the urethra 910 (between the urethra 910 and the vagina 920), and the obturator foramen 932, and to exit the living body via the body surface in an inguinal region on the right side or at a part in the vicinity of this inguinal region. As a result, the patient is formed therein with a puncture hole 940 for embedding the implant 300 inside the living body.

Note that the puncture needle 210 can be expressed in the form of an image and displayed on the display unit 23, by the image data forming apparatus 1. Therefore, the puncture by the puncture needle 210 can be performed while checking whether the needle tip of the puncture needle 210 displayed on the display unit 23 is being advanced along the puncture path L. Consequently, safety can further be enhanced.

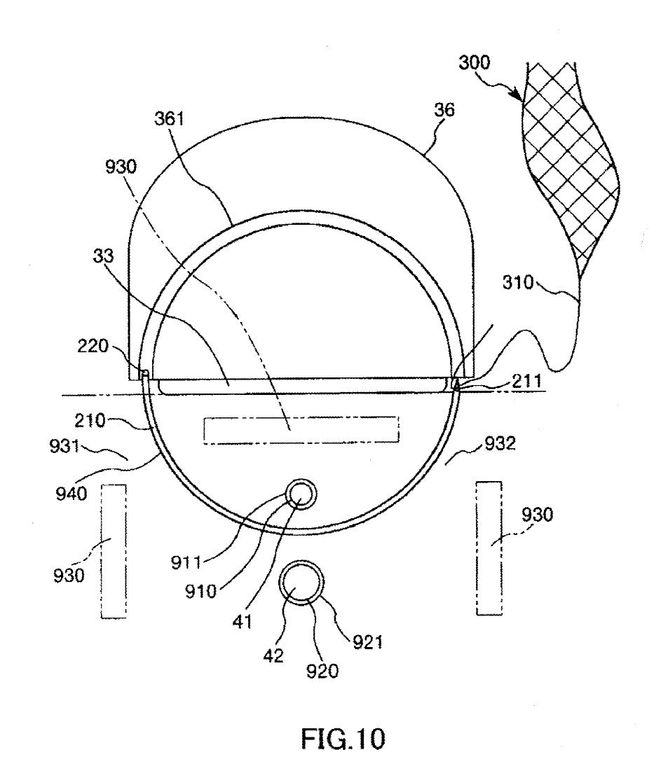

Next, as shown in FIG. 10, the string 310 fixed to the implant 300 is inserted into and passed through the through-hole 211 of the puncture needle 210. As a result, the string 310 is retained at the distal portion of the puncture needle 210. Subsequently, with the operation unit 220 grasped, the puncture member 200 is rotated clockwise in FIG. 10. By this operation, the needle tip of the puncture needle 210 is made to enter the living body via the body surface in the inguinal region on the right side of the patient or at the part in the vicinity of the inguinal region, to sequentially pass the obturator foramen 932, between the urethra 931 and the vagina 920, and the obturator foramen 931, and to exit the living body via the body surface in the inguinal region on the left side or at the part in the vicinity of this inguinal region. In accordance with an exemplary embodiment, for example, the puncture needle 210 is drawn out of the living body. Next, the string 310 is drawn out of the through-hole 211, and the urethral ultrasonic marker 41, the vaginal ultrasonic marker 42, the puncture member 200 and the puncture needle guiding device 3 are removed from the patient.

Subsequently, the string 310 is pulled to insert the implant 300 into the puncture hole 940, and the implant 300 is embedded in the living body, leaving both end portions of the implant 300 exposed outside of the living body. Next, the string 310 and the string 320 are pulled to adjust the position of the implant 300 relative to the urethra 910, and unnecessary portions of the implant 300 are cut away. Finally, predetermined treatments are conducted to finish the procedure.

According to the using method as above, the puncture by the puncture needle 210 can be performed on a visible basis by use of the image data forming apparatus 1. Therefore, the puncturing by the puncture needle 210 can be carried out relatively accurately and safely, independently of the operator's experiences or skill. In addition, since the puncture needle 210 is guided by the guide groove 361 at the time of puncturing by the puncture needle 210, the puncture by the puncture needle 210 can be performed relatively smoothly. In addition, since the puncture by the puncture needle 210 can be conducted relatively smoothly, the puncture by the puncture needle 210 can be performed with a reduced force.

A second embodiment of the puncture needle guiding device and the image data forming apparatus of the present disclosure will be described below.

The image data forming apparatus in the second exemplary embodiment will be described below, referring primarily to differences from the aforementioned first embodiment and while omitting descriptions of the same items as above.

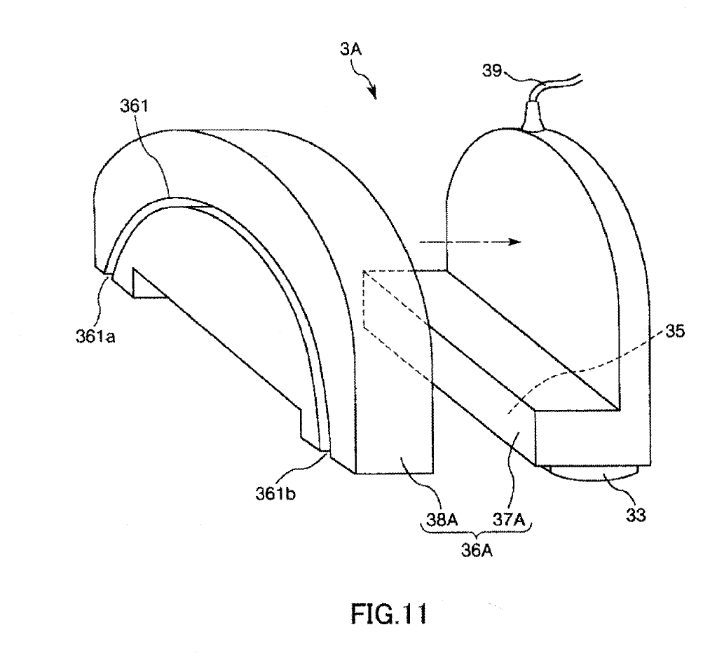

As shown in FIG. 11, a puncture needle guiding device 3A in this embodiment has a configuration in which a guide groove 361 is freely detachable in relation to an ultrasound transmission-reception unit 35. Specifically, a casing 36A can include a first casing 37A accommodating the ultrasound transmission-reception unit 35, and a second casing (a guiding tool) 38A formed with the guide groove 361, wherein the second casing 38A is freely detachable in relation to the first casing 37A. By thus making the second casing 38A freely detachable in relation to the first casing 37A, the second casing 38A can be mounted to an existing ultrasonic probe, for example. In addition, although it may become necessary to modify the guide groove 361 depending on the size and shape of a puncture needle 210 to be used, such a situation can be coped with by selecting an optimum second casing 38A from among a plurality of second casings 38A provided respectively with different-shaped guide grooves 361 and mounting the selected second casing 38A to the first casing 37A. Consequently, a highly convenient device can be realized.

Note that the positional relation between the guide groove 361 and the ultrasound transmission-reception unit 35 in the state where the second casing 38A has been fixed to the first casing 37A is the same as in the aforementioned first embodiment. In addition, the method for fixing the first and second casings 37A and 38A to each other is not specifically restricted, and any mechanism such as, for example, screwing or fitting may be used.

According to the second embodiment as above, also, the same or equivalent effects to those of the aforementioned first embodiment can be produced.

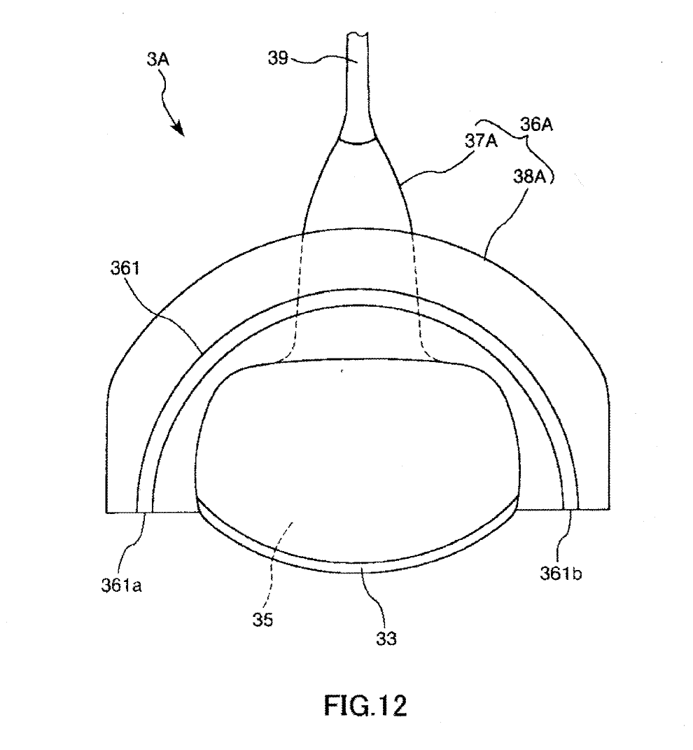

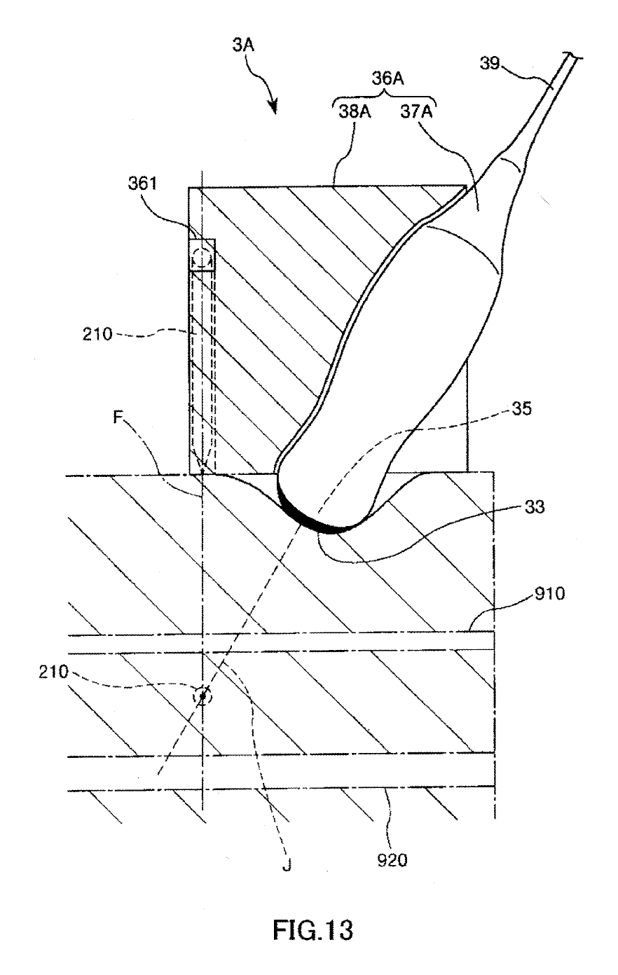

Note that the shapes of the first casing 37A and the second casing 38A are not particularly limited. For example, the shapes may be as shown in FIG. 12 or as shown in FIG. 13.

In accordance with an exemplary embodiment, for example, in the case of the shapes as shown in FIG. 13, a guide groove 361 is disposed at a position deviated from an acoustic lens 33, and a plane (a plane containing a puncture path for a needle tip of a puncture needle 210) F that contains the guide groove 361 is inclined against an ultrasound transmission axis J. The puncture needle guiding device 3A is so configured that the transmission axis J intersects the plane F between the urethra 910 and the vagina 920 in the state shown in FIG. 13 (the state in use), which can help ensure that a puncture path L for the puncture needle 210 passing between the urethra 910 and the vagina 920 can be reliably expressed in the form of an image. Therefore, the puncture by the puncture needle 210 can be performed on a visible basis and in a relatively accurate and safe manner. Note that while the configuration wherein the transmission axis J intersects the plane F between the urethra 910 and the vagina 920 in the state in use as aforementioned is adopted in this embodiment, this configuration is not restrictive, so long as the puncture path for the puncture needle 210 can be expressed in the form of an image.

A third exemplary embodiment of the puncture needle guiding device and the image data forming apparatus of the present disclosure will be described below.

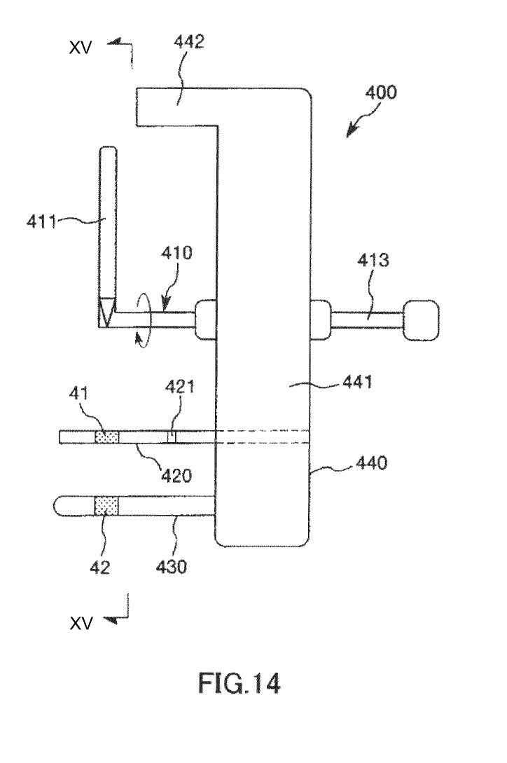

In the following, the left side in FIG. 14 will be referred to as “distal” side, the right side as “proximal” side, the upper side as “upper” side, and the lower side as “lower” side.

The third embodiment will be described below, referring mainly to differences from the aforementioned first embodiment and while omitting descriptions of the same items as above.

A puncture needle guiding device 3 in this embodiment is configured to be interlockable to a puncture apparatus 400.

As shown in FIG. 14, the puncture apparatus 400 can include a puncture member 410 which punctures biological tissue; an elongated urethral-insertion member (insertion section) 420 to be inserted into a urethra; an elongated vaginal-insertion member (insertion section) 430 to be inserted into a vagina; and a support member 440 supporting the puncture member 410, the urethral-insertion member 420 and the vaginal-insertion member 430.

The urethral-insertion member 420 is fixed to the support member 440. The urethral-insertion member 420 is straight bar-like in shape, and its proximal opening is opening in a proximal surface of the support member 440. In the urethral-insertion member 420, there can be inserted any of various elongated medical instruments. Examples of the medical instrument include a balloon catheter provided at a distal portion thereof with a balloon, which can be expanded and contracted. The balloon of the balloon catheter functions as a restriction unit, which restricts the position in the axial direction of the urethral-insertion member 420 within the urethra. Specifically, when the puncture apparatus 400 is used, the balloon is inserted into the patient's bladder and then expanded there. As a result, the balloon is caught on the bladder neck, whereby the position of the urethral-insertion member 420 relative to the bladder and the urethra is fixed.

The urethral-insertion member 420 is provided with a marker 421 at a peripheral portion thereof. The marker 421 is so disposed that it is positioned at the urethral orifice when the urethral-insertion member 420 is inserted in the urethra and a distal portion of the urethral-insertion member 420 is positioned just on the operator's side of the bladder. In addition, a urethral ultrasonic marker 41 is provided at an intermediate portion of the urethral-insertion member 420.

The vaginal-insertion member 430 is secured to the support member 440. The vaginal-insertion member 430 is straight rod-like in shape. A distal end portion of the vaginal-insertion member 430 is rounded, which helps enable smooth insertion of the vaginal-insertion member 430 into the vagina. In addition, the vaginal-insertion member 430 is provided with a vaginal ultrasonic marker 42 at an intermediate portion thereof. The vaginal ultrasonic marker 42 is provided oppositely to the urethral ultrasonic marker 41, and a needle tip of a puncture needle 411 passes a region between these markers 41 and 42. The vaginal-insertion member 430 is disposed on the lower side of the urethral-insertion member 420, at a predetermined separated distance from the urethral-insertion member 420, so that its axis and the axis of the urethral-insertion member 420 are parallel to each other.

The materials constituting the vaginal-insertion member 430, the urethral-insertion member 420, and the support member 440 are not particularly limited; for example, various metallic materials, various resin materials and the like can be used.

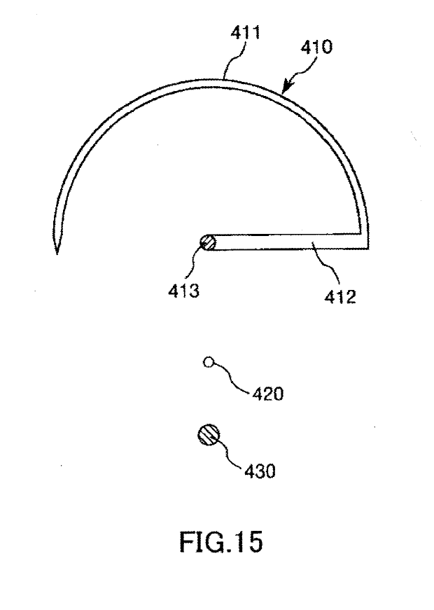

As shown in FIG. 15, the puncture member 410 can include an arc-shaped puncture needle 411 which punctures biological tissue; a shaft section 413; an interlock portion 412 interlocking the puncture needle 411 and the shaft section 413; and an operation unit 414 provided at a proximal portion of the shaft section 413. The puncture needle 411 is configured in the same manner as the puncture needle 210 in the aforementioned first embodiment. The shaft section 413 extends in a direction which intersects the center of the puncture needle 411 and which is orthogonal to a plane containing the puncture needle 411.

The puncture member 410 as above is borne on the support member 440 at the shaft section 413, whereby the puncture member 410 can be rotationally moved about the shaft section 413 in relation to the support member 440. In accordance with an exemplary embodiment, for example, the shaft section 413 (rotating shaft) of the puncture member 410 constitutes a guide section for the puncture needle 411. As a result, movements of the puncture needle 411 in unintended directions can be inhibited, and the puncture needle 411 can be guided relatively smoothly. Note that an operation to rotate the puncture member 410 can be carried out relatively easily by operating the operation unit 414.

The shaft section 413 is disposed on the upper side of the urethral-insertion member 420, at a predetermined separated distance from the urethral-insertion member 420, so that the axis of the shaft section 413 is parallel to the axis of the urethral-insertion member 420. In addition, the shaft section 413, the urethral-insertion member 420, and the vaginal-insertion member 430 are disposed on a straight line, when viewed from the axial direction of the shaft section 413.

The support member 440 can include a main body 441 which supports the puncture member 410, the urethral-insertion member 420 and the vaginal-insertion member 430; and an interlock portion 442 for interlocking the main body 441 to a third casing 36 of the puncture needle guiding device 3.

The main body 441 can determine the positional relation between the urethral-insertion member 420 and the vaginal-insertion member 430 so that, when the puncture member 410 is rotationally moved to cause the puncture needle 411 to puncture biological tissue, the needle tip of the puncture needle 411 passes between the urethral-insertion member 420 or its extension line and the vaginal-insertion member 430 or its extension line.

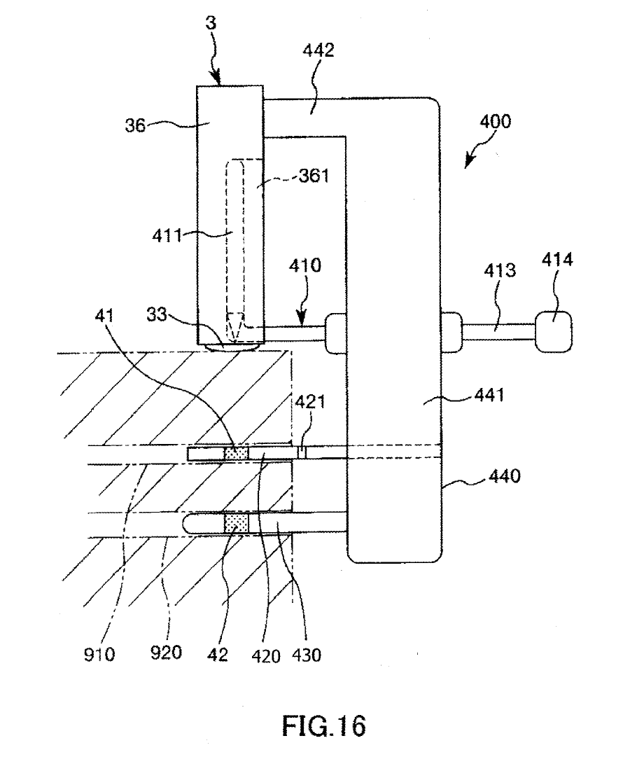

As shown in FIG. 16, in a state in which the interlock portion 442 is interlocked to the casing 36, the puncture needle 411 is disposed in a guide groove 361 formed in the casing 36. Note that the method for interlocking the casing 36 and the interlock portion 442 is not particularly limited, and any interlocking mechanism such as, for example, screwing or fitting can be used.

According to the puncture apparatus 400 as above, an implant 300 can be embedded into a living body, like in the aforementioned first embodiment, by a method wherein the puncture member 410 is rotated to cause the puncture needle 411 to puncture the living body in a state in which the urethral-insertion member 420 is inserted in the urethra 910 and the vaginal-insertion member 430 is inserted in the vagina 920, as shown in FIG. 16. In this instance, by interlocking the puncture needle guiding device 3 to the puncture apparatus 400, the positional relation between the urethral ultrasonic marker 41 and the vaginal ultrasonic marker 42 can be kept constant. Therefore, the urethra and the vagina can constantly be captured on a display unit 23 by the puncture needle guiding device 3. Consequently, the speediness and safety of the procedure can be enhanced. In addition, since the puncture needle guiding device 3 is stably supported by the urethral-insertion member 420 and the vaginal-insertion member 430, the guiding function for the puncture needle 411 is further enhanced. Thus, by using the puncture needle guiding device 3 in cooperation with the puncture apparatus 400, the puncture by the puncture needle 210 can be performed safely. Note that the term “cooperation” means that the puncture needle guiding device 3 and the puncture apparatus 400 cooperate with each other, by exhibiting their functions in combination, in placing the implant 300 indwelling inside a living body.

According to the third embodiment as above, also, the same or equivalent effects to those of the aforementioned first embodiment can be produced.

Note that while the urethral-insertion member 420 and the vaginal-insertion member 430 are used as insertion sections in this embodiment, this is not restrictive of the configuration of the insertion section. For example, either one of the urethral-insertion member 420 and the vaginal-insertion member 430 may be omitted. In addition, the insertion section is not limited to the one to be inserted into a body lumen or cavity such as the urethra and the vagina, and may be any one that is inserted or introduced into a living body. Specifically, for example, the insertion section may be composed of an ultrasonically visible implant capable of being set indwelling in the urethra or the vagina, an ultrasonic-basis contrast medium composing of a liquid or a gas, or the like.

In this embodiment, the puncture needle guiding device 3 is provided so as to be interlockable to the puncture apparatus 400, and they are used in the interlocked state. However, they may not necessarily be interlocked to each other, so long as the puncture needle guiding device 3 can be used in cooperation with the puncture apparatus 400. Specifically, for example, the puncture needle guiding device 3 may be used in a state in which the posture of the puncture needle guiding device 3 relative to the puncture apparatus 400 (the urethral-insertion member 420 and the vaginal-insertion member 430) can be freely changed. Even where the puncture needle guiding device 3 and the puncture apparatus 400 are not interlocked to each other, the same or equivalent effects to those of this embodiment can be produced by using the puncture needle guiding device 3 in cooperation with the puncture apparatus 400. In the case where the puncture needle guiding device 3 and the puncture apparatus 400 are used in a non-interlocked state, the puncture apparatus 400 (the urethral-insertion member 420 and the vaginal-insertion member 430) corrects the postures (shapes) of the urethra and the vagina so that a space sufficient for passing the puncture needle 210 therethrough can be secured between the urethra and the vagina, and the puncture needle guiding device 3 can guide the puncture needle 411 into and through a region between the urethra 910 and the vagina 920, whereby the placement of the implant 300 indwelling inside the living body can be performed relatively safely.

In addition, while the puncture needle guiding device 3 can be interlocked to the puncture apparatus 400 in this embodiment, the puncture needle guiding device 3 may be formed to be integral with the puncture apparatus 400. In accordance with an exemplary embodiment, for example, the puncture needle guiding device 3 and the puncture apparatus 400 may not be formed in a freely detachable state.

In addition, while the shaft section 413 and the guide groove 361 are provided as the guide section for guiding the puncture needle 411 in this embodiment, one of the shaft section 413 and the guide groove 361 may be omitted.

While the puncture needle guiding device and the image data forming apparatus of the present disclosure have been described referring to the illustrated embodiments above, the present disclosure is not limited to the embodiments. Each of the components of the puncture needle guiding device and the image data forming apparatus may be replaced by an arbitrary component capable of exhibiting the same or equivalent function to the original. In addition, an arbitrary structure or structures may be added to the aforementioned configurations. In addition, the puncture needle guiding device and the image data forming apparatus of the present disclosure may each be a combination of arbitrary two or more configurations (features) of the above embodiments.

In the above embodiments, description has been made of the case where the puncture needle guiding device and the image data forming apparatus of the present disclosure are applied to a device for use in embedding in a living body an embeddable implant for treatment of female urinary incontinence. The use of the puncture needle guiding device and the image data forming apparatus of the present disclosure, however, is not limited to the described one.

For example, the target of the application of the present disclosure can include excretory disorders attendant on the weakening of the pelvic floor muscle group (urinary urgency, frequent urination, urinary incontinence, fecal incontinence, urinary retention, dysuria or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues include bladder, vagina, uterus, bowel and the like. Lessactive tissues include bones, muscles, fascias, ligaments and the like. In particular, for example, in the case of pelvic floor disorders, the lessactive tissues include an obturator fascia, a coccygeus fascia, a cardinal ligament, an uterosacral ligament, and a sacrospinous ligament.

For the procedure for interlocking an overactive tissue in the pelvic floor disorder with the lessactive tissue, there are included a retropubic sling surgery, a transobturator sling surgery, transobturator tape (TOT) surgery, a tension-free vaginal mesh (TVM) surgery, a uterosacral ligament suspension (USLS) surgery, a sacrospinous ligament fixation (SSLF) surgery, an iliococcygeus fascia fixation surgery, and a coccygeus fascia fixation surgery.

The puncture needle guiding device of the present disclosure can include a casing; an ultrasound transmission-reception unit which is disposed inside the casing and which transmits and receives ultrasound; and a guide section which is provided in the casing and which guides a curved puncture needle adapted to puncture a subject body. Therefore, the puncture needle can be guided by the guide section and, accordingly, the puncture by the puncture needle can be performed relatively smoothly. In addition, since the puncture by the puncture needle can be conducted relatively smoothly, the puncture by the puncture needle can be carried out with a reduced force, so that the safety in the surgical treatment can be enhanced. In addition, where the image based on the information obtained by the ultrasound transmission-reception unit is displayed on a display unit such as, for example, a monitor, the puncture by the puncture needle can be performed on a visible basis, which can help enable the puncture by the puncture needle to be carried out relatively accurately and safely. Particularly, for example, where a puncture path for the needle tip of the puncture is displayed in the state of being superimposed on an ultrasonic image displayed on the monitor, the puncture needle guiding device can be easily disposed in a suitable position, so that the puncture by the puncture needle can be conducted relatively accurately and safely.

Thus, the puncture needle guiding device of the present disclosure has industrial applicability.

The detailed description above describes puncture needle guiding device and an image data forming apparatus. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A puncture needle guiding device comprising:
a casing;
an ultrasound transmission-reception unit which is disposed inside the casing and which transmits and receives ultrasound; and
a guide section in the casing and which guides a curved puncture needle adapted to puncture a subject body.

2. The puncture needle guiding device according to claim 1, wherein the puncture needle is rotationally movable.

3. The puncture needle guiding device according to claim 1, wherein the puncture needle is rotationally movable so as to describe an arc.

4. The puncture needle guiding device according to claim 1, wherein the puncture needle is arc-shaped.

5. The puncture needle guiding device according to claim 1, wherein the guide section includes a groove.

6. The puncture needle guiding device according to claim 5, wherein the groove includes a pair of restriction units, which are located on both sides in a width direction of the puncture needle.

7. The puncture needle guiding device according to claim 1, wherein the guide section is freely detachable from the casing.

8. An image data forming apparatus comprising:
a puncture needle guiding device, the puncture needle guiding device including:
a casing;
an ultrasound transmission-reception unit which is disposed inside the casing and which transmits and receives ultrasound; and
a guide section in the casing and which guides a curved puncture needle adapted to puncture a subject body; and
a control unit which forms image data for displaying on a display unit an image based on information obtained by the ultrasound transmission-reception unit.

9. The image data forming apparatus according to claim 8, wherein the control unit forms image data for displaying on the display unit both the image based on the information obtained by the ultrasound transmission-reception unit and an image including a puncture path for a needle tip of the puncture needle.

10. The image data forming apparatus according to claim 8, wherein the image data further includes:
a positioning marker for positioning the puncture needle guiding device such that when a body lumen or cavity and the positioning marker are matched to each other on the display unit, the puncture needle is guided by the guide section so as to avoid the body lumen or cavity.

11. The image data forming apparatus according to claim 8, wherein the casing is used in cooperation with an insertion section which is inserted into a living body or into a body lumen or cavity.

12. The image data forming apparatus according to claim 8, wherein the ultrasound transmission-reception unit is used for treatment of a disorder in a pelvic organ and is so disposed as to make contact with a body surface.

13. The image data forming apparatus according to claim 8, wherein the guide section includes a groove.

14. The image data forming apparatus according to claim 13, wherein the groove includes a pair of restriction units, which are located on both sides in a width direction of the puncture needle.

15. The image data forming apparatus according to claim 8, wherein the guide section includes a rotating shaft of the puncture needle which is rotationally movable.

16. The image data forming apparatus according to claim 11,
wherein the body lumen or cavity is a urethra; and
the puncture needle guiding device is so configured that the insertion section and the casing can be interlocked to each other.

17. A method of guiding a puncture needle during formation of a path in a living body, the method comprising:
transmitting and receiving ultrasound from an ultrasound transmission-reception unit which is disposed inside a casing;
guiding a curved puncture needle with a guide section in the casing; and
puncturing the living body with the puncture needle.

18. The method according to claim 17, comprising:
rotationally moving the puncture needle in an arc.

19. The method according to claim 17, comprising:
wherein the guide section includes a groove; and
restricting movement of puncture needle within the groove with a pair of restriction units, which are located on both sides in a width direction of the puncture needle.

20. The method according to claim 17, comprising:
positioning the puncture needle guiding device using a positioning marker, such that when a body lumen or cavity and the positioning marker are matched to each other on a display unit based on an image based on information obtained by the ultrasound transmission-reception unit, the puncture needle is guided by the guide section so as to avoid the body lumen or cavity.
